(19) United States
(12) Patent Application Publication
(10) Pub. No.: US 2009/0281110 A1
(43) Pub. Date: Nov. 12, 2009

(54) Method of Treatment

(76) Inventors: Jakob Busch-Petersen, King of Prussia, PA (US); Jeffery D. Bray, King of Prussia, PA (US); Nicholas J. Laping, King of Prussia, PA (US); Eugene T. Grygielko, King of Prussia, PA (US); Richard M. Goodman, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/306,024

(22) PCT Filed: Jun. 22, 2007
(86) PCT No.: PCT/US07/71864

§ 371 Date: Dec. 22, 2008

Related U.S. Application Data
(60) Provisional application No. 60/805,626, filed on Jun. 23, 2006.

(51) Int. Cl. A61K 31/495

(52) U.S. Cl. 514/252.12

(57) ABSTRACT

This invention relates to the use of sulfonamide substituted diphenyl urea compounds to treat endometriosis.

### FIELD OF THE INVENTION

This invention relates to the use of sulfonamide substituted diphenyl urea compounds to treat IL-8, GROα, GROβ, GROγ, NAP-2, and ENA-78 mediated diseases, particularly endometriosis.

### BACKGROUND OF THE INVENTION

Endometriosis is a disease characterized by the growth of endometrial tissue (called lesions) at extrauterine sites. This lesion attachment can result in pain, dysmenorrhea, dyspareunia, and infertility. It is estimated that greater than 80% of patients presenting with chronic pelvic pain are eventually diagnosed with endometriosis. The prevalence of the disease is about 7-10% of women of reproductive years, with a familial association risk increase of ten-fold. Definitive diagnosis is only reached by laparoscopy, but typically there is about a ten year delay from disease onset to conclusive diagnosis. Consistent with their uterine origins, the endometriotic lesions are hormonally dependent upon estrogen. Therapies that functionally antagonize estrogen production or action, such as progestins and Gonadotropin Releasing Hormone (GnRH) analogues, are efficacious in alleviating symptoms and reducing the extent of the disease. Current therapeutic goals include reducing pain with anti-inflammatory agents and suspending the ovarian cycle using hormonal modulation drugs.

All of these therapies have therapeutic limitations specific for the mechanisms of action. Though progestins are used to treat endometriosis, these agents cause a number of adverse effects, including breakthrough bleeding, mood alteration, acne, weight gain, and breast tenderness. The GnRH analogues induce a hypoestrogenic state with adverse effects including bone loss and vasomotor symptoms. Hormonal modulation is contra-indicating for fertility, which is, paradoxically, why many patients seek treatment for endometriosis. The anti-inflammatory agents administered tend to be NSAIDs or Cyclo-oxygenase 2-selective (COX-2) inhibitors. The nonselective NSAIDs have adverse gastro-intestinal effects, while COX-2 inhibitors have increased risk for adverse cardiovascular effects. There is substantial evidence that endometriosis is also an immunological disease and associated with high levels of cytokines, chemokines, macrophages and neutrophils. Cytokine/chemokine levels in many cases correlate with disease severity. Therefore, immunomodulatory therapy with enhanced selectivity towards endometriosis, while preserving fertility, is highly desired.

Chemokines are produced by endometrial and other uterine cell types and the IL-8:CXCR2 signaling pathway is particular relevant to normal physiology and endometriosis. IL-8 expression is cyclical with highest levels during the late secretory and menstruation phases of the menstrual cycle, consistent with the highest IL-8 levels in menstrual debris that moves into the peritoneal cavity by retrograde action. CXCR2 is a receptor for IL-8 and is expressed in both endometrial epithelium and stroma, with highest levels in apical epithelium. This pattern is similar to the expression pattern for IL-8. CXCR2 is significantly increased in ectopic endometriotic tissue and adenomyosis. IL-8 expression is higher in ectopic endometrial cells compared to eutopic endometrial cells. Furthermore, IL-8 in peritoneal fluid and sera correlates with endometriosis disease stage and infertility.

Other CXCR2 ligands also have been implicated in endometriosis. Peritoneal fluid concentrations of epithelial neutrophil-activating peptide-78 (ENA-78) correlate with the severity of endometriosis. The expression of IL-8 and its cognate receptor, CXCR2, in normal endometrial tissues, coupled with the apparent dysregulation in endometriosis, suggest that inhibition of CXCR2 may provide a therapeutic benefit to women with endometriosis.

### SUMMARY OF THE INVENTION

The present invention relates to a method of treating endometriosis in a woman diagnosed as having endometriosis. The method involves administering to the woman having endometriosis, N-[4-chloro-2-hydroxy-3-(piperazine-1-sulfonyl)phenyl]-N′-(2-chloro-3-fluorophenyl)urea, or a pharmaceutically acceptable salt thereof, in an amount effective to reduce the size of endometriotic tissue in the woman.

The present invention also relates to a method of preventing endometriosis in a woman at higher than normal risk of developing or suffering recurrence of endometriosis. The method involves administering to the woman, N-[4-chloro-2-hydroxy-3-(piperazine-1-sulfonyl)phenyl]-N′-(2-chloro-3-fluorophenyl)urea, or a pharmaceutically acceptable salt thereof, in an amount effective to reduce or prevent the growth or thickening of endometriotic tissue in the woman.

### DETAILED DESCRIPTION OF THE INVENTION

This invention provides for a method of treating endometriosis comprising administering to a mammal, in particular a human female, in need thereof an effective amount of N-[4-chloro-2-hydroxy-3-(piperazine-1-sulfonyl)phenyl]-N′-(2-chloro-3-fluorophenyl)urea or a pharmaceutically acceptable salt thereof. The method involves administering to a woman having endometriosis or to a woman at higher than normal risk of developing endometriosis, a dose of N-[4-chloro-2-hydroxy-3-(piperazine-1-sulfonyl)phenyl]-N′-(2-chloro-3-fluorophenyl)urea, or a pharmaceutically acceptable salt thereof, in an amount sufficient to reduce the size or inhibit the growth or thickening of endometriotic tissue in the woman.

For the purposes of the present invention, successful treatment of endometriosis in accordance with the inventive method encompasses, but is not limited to, reducing the size of endometriotic tissue present in the woman. In accordance with the inventive method “reducing the size” of endometriotic tissue encompasses reducing the mass or weight, diameter, length, width, circumference, and/or thickness or height, of the endometriotic tissue. Also included as indicative of successful treatment in accordance with the inventive method are detectable improvements in symptoms of endometriosis, for example, a pregnancy and/or a reduction in pelvic pain experienced by the woman, regardless of whether the size of her endometriotic tissue is actually measured.

For the purposes of the present invention, preventing endometriosis encompasses inhibiting or reducing the size of endometriotic tissue present in the woman and/or preventing the development symptoms of endometriosis, regardless of whether the size of her endometriotic tissue is actually measured.

In accordance with the present invention, a woman is a female human post-menarche, including pubescent and adult women having periodic menses, menopausal women, and post-menopausal women. A woman having endometriosis refers to a woman medically diagnosed with endometriosis. Endometriosis is a condition in which abnormal formations of endometriotic tissue develop in locations other than the uterus. Endometriotic tissue resembles endometrium and responds to estrogen by thickening. Typically, the diagnosis of endometriosis is done by surgical means, such as laparoscopy or laparotomy, involving direct observation of the endometriotic tissue. However, other medically accepted means of diagnosis are contemplated for the purposes of the present invention. Clinical symptoms of endometriosis can also contribute to the diagnosis of the condition. Symptoms commonly include infertility and pelvic pain, low sacral backaches, bloody urine or stool, pain or bleeding with defecation, urination, or intercourse, pelvic discomfort or pressure, and premenstrual spotting.

A woman at higher than normal risk of developing endometriosis is a woman at greater risk than the general population of women of developing endometriosis for the first time or suffering a recurrence of endometriosis. This does not mean that, untreated, the woman at higher risk of developing endometriosis will certainly develop the condition, merely that her aggregated risk factors are greater than average. Known risk factors for endometriosis include early menarche (before age 13 years), frequent menstruations (e.g. menstrual cycles of 27 days or less), unusually long menstrual periods (5-7 days or longer), chronic pelvic pain, especially with stenosis of external cervical os, advanced age, Asian race, the presence of Mullerian anomalies (e.g. duplicate cervix and vagina), long duration of uninterrupted menstrual cycles, long duration of intrauterine device (IUD) use, infertility, nulliparity, only one live birth, or after ten years of the last birth.

Women having had cervical conization or gynecological laparotomies, ovarian surgeries, or hysterectomies are also at higher than normal risk for endometriosis. Women who have used oral contraceptives are also at higher than normal risk of developing endometriosis. Familial risk factors can also contribute to a higher than normal risk of developing endometriosis, such as a sibling, mother, aunt or cousin having been diagnosed with endometriosis.

The preceding is merely illustrative of factors that can contribute to a woman being at higher than normal risk of developing endometriosis, and is not an exhaustive list.

According to the instant method, women with endometriosis or those at risk for developing endometriosis, are treated with N-[4-chloro-2-hydroxy-3-(piperazine-1-sulfonyl)phenyl]-N′-(2-chloro-3-fluorophenyl)urea, or a pharmaceutically acceptable salt thereof.

The compound used in the method of the invention can be administered as a pharmaceutically acceptable salt. Suitable pharmaceutically acceptable salts are well known to those skilled in the art and include salts of inorganic and organic acids, such as hydrochloric acid, hydrobromic acid, sulfuric acid, phosphoric acid, methane sulfonic acid, ethane sulfonic acid, toluenesulfonic acid, acetic acid, malic acid, tartaric acid, citric acid, lactic acid, oxalic acid, succinic acid, fumaric acid, maleic acid, benzoic acid, salicylic acid, phenylacetic acid and mandelic acid.

In addition, pharmaceutically acceptable salts may also be formed with a pharmaceutically acceptable cation. Suitable pharmaceutically acceptable cations are well known to those skilled in the art and include alkaline, alkaline earth, ammonium and quaternary ammonium cations.

In one embodiment, the hydrochloric acid salt of N-[4-chloro-2-hydroxy-3-(piperazine-1-sulfonyl)phenyl]-N′-(2-chloro-3-fluorophenyl)urea is a compound used in the method of this invention.

In another embodiment, the p-toluenesulfonic acid salt of N-[4-chloro-2-hydroxy-3-(piperazine-1-sulfonyl)phenyl]-N′-(2-chloro-3-fluorophenyl)urea is a compound used in the method of this invention.

### EXAMPLES

Nuclear magnetic resonance spectra were recorded at either 300 or 400 MHz using, respectively, a Bruker ARX 300 or Bruker AVANCE 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (Δ) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Fourier transform infrared (FTIR) spectra were recorded on a Nicolet 510 infrared spectrometer. FTIR spectra were recorded in transmission mode, and band positions are reported in inverse wavenumbers (cm⁻¹). Mass spectra were taken on either a SCIEX5 or Micromass instruments, using electrospray (ES) ionization techniques. Elemental analyses were obtained using a Perkin-Elmer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel. Analytical and preparative HPLC were carried out on Rainin or Beckman chromatographs. ODS refers to an octadecylsilyl derivatized silica gel chromatographic support. 5μ Apex-ODS indicates an octadecylsilyl derivatized silica gel chromatographic support having a nominal particle size of 5μ, made by Jones Chromatography, Littleton, Colo. YMC ODS-AQ® is an ODS chromatographic support and is a registered trademark of YMC Co. Ltd., Kyoto, Japan. PRP-1® is a polymeric (styrene-divinylbenzene) chromatographic support, and is a registered trademark of Hamilton Co., Reno, Nev.) Celite® is a filter aid composed of acid-washed diatomaceous silica, and is a registered trademark of Manville Corp., Denver, Colo.

The following Examples are intended to be illustrative only and not limiting in any way.

### Example 1

### N-[4-chloro-2-hydroxy-3-(piperazine-1-sulfonyl)phenyl]-N′-(2-chloro-3-fluorophenyl)urea hydrochloride

### 1a) 2-chloro-3-fluorobenzoic acid

A solution of 3-fluorobenzoic acid (4.02 g, 28.71 mmol) in 20 mL of THF was added dropwise to a suspension of tetramethylenediamine (TMEDA) (10.00 mL, 66.3 mmol) and 1.3 M sec-BuLi (48 mL, 62.4 mmol) in 50 mL of THF at −90° C. The mixture was stirred at −90° C. for 35 min. The mixture was warmed to −78° C. when a solution of hexachloroethane (27.0 g, 113.9 mmol) in 50 mL of THF was added. After 20 h, the reaction was quenched with water and diluted with diethyl ether. The bilayer was adjusted to pH ˜1-2 with conc. HCl. The organic layer was washed with water, brine, dried and concentrated to give 30.4 g crude as a tan solid, which was washed with hexane to give 3.728 g (74%) of the desired product 1a (light tan solid). MS (m/z) 175.2 (M+H).

### 1b) 3-chloro-2-fluoro-benzoyl azide

A suspension of 2-chloro-3-fluorobenzoic acid (2.704 g, 15.54 mmol) in 25 mL of oxalyl chloride was heated to reflux for 2 h. The solution was cooled and concentrated to give the crude acid chloride 3.13 g as a brown liquid which was directly used in the next step. A solution of NaN₃ (2.79 g, 43 mmol) in 10 mL of water was added dropwise to a solution of the crude acid chloride (3.13 g) in 20 mL acetone at 0° C. After 15 min, the solution was diluted with CH₂Cl₂ and washed with water and brine. The organic layer was dried and concentrated to give a brown liquid which was filtered through silica gel using ethyl acetate/hexane (5/95, v/v) to yield 2.97 g (96%) of 1b (colorless liquid). The compound was used without further purification.

### 1c) 2-(2-tert-Butyl-6-chloro-benzooxazole-7-sulfonyl)-piperazine-1-carboxylic acid tert-butyl ester

The solution of 2-tert-butyl-6-chloro-benzooxazole-7-sulfonyl chloride (10.75 g, 34.9 mmol) in 100 mL of THF was cooled to 0° C., Et₃N (3.47 mL, 24.9 mmol) and then Boc-piperazine (5.0 g, 26.8 mmol) were added. The resulting mixture was stirred for 20 h, warming to room temperature. The mixture was poured into water, extracted with EtOAc and washed with another portion of water; organic layers were dried and concentrated. Purification by column chromatography on silica gel, eluting with ethyl acetate/hexane (30/70, v/v), yielded 11.12 g (91%) desired product 1c. LC-MS (m/z) 458.2 (M+H).

### 1d) 4-(3-Amino-6-chloro-2-hydroxy-benzenesulfonyl)-piperazine-1-carboxylic acid tert-butyl ester

The solution of starting material 1e (1.12 g) in dioxane (20 mL) was treated with water (11 mL) and concentrated H₂SO₄ (11 mL). The mixture was heated to reflux for 12 h. The reaction mixture was concentrated and then basified the residue to pH ˜14 with 50% aq NaOH. (Boc)₂O (5.6 g, 1.05 eq) and 100 mL of AcOEt were added, the resulting mixture was stirred at room temperature for 16 h. The mixture was separated, the water layer was extracted with EtOAc, organic layers were combined, dried and concentrated. Purification by column chromatography on silica gel, eluting with ethyl acetate/hexane (30/70, v/v), yielded 8.18 g (85%) desired product 1d. ¹H NMR (CDCl₃): δ 6.85 (m, 2H), 3.48 (t, 4H), 3.25 (t, 4H), 1.47 (s, 9H).

### 1e) 4-(6-chloro-3-[3-(2-chloro-3-fluorophenyl)-ureido]-2-hydroxy-benzenesulfonyl)-piperazine-1-carboxylic acid tert-butyl ester

A solution of 4-(3-amino-6-chloro-2-hydroxy-benzenesulfonyl)-piperazine-1-carboxylic acid tert-butyl ester (3.8 g, 9.7 mmol) and 3-chloro-2-fluorobenzoyl azide (2.9 g, 14.5 mmol) in 5 mL of N,N-dimethylformamide was stirred at room temperature for 18 h. The mixture was diluted with ethyl acetate and washed with water to give the crude material. Purification by column chromatography on silica gel, eluting with ethyl acetate/hexane (20/80, v/v), gave 3.6 g (66%) of 1e. LC-MS (m/z) 562.8 (M+H).

### 1f) N-[4-chloro-2-hydroxy-3-(4-methyl-piperazine-1-sulfonyl)-phenyl]-N′-(2-chloro-3-fluorophenyl)urea hydrochloride

A solution of 3.6 g Boc-product (1 g) in 20 mL of 4N HCl in dioxane was stirred at room temperature for 2 h and the solvent was evaporated. The residue was recrystallized from methanol and ethyl acetate to give the title product 2.9 g, (60%). LC-MS (m/z) 463.0 (M+H).

### Example 2

### N-[4-chloro-2-hydroxy-3-(4-methyl-piperazine-1-sulfonyl)-phenyl]-N′-(2-chloro-3-fluorophenyl)-urea p-toluenesulfonate

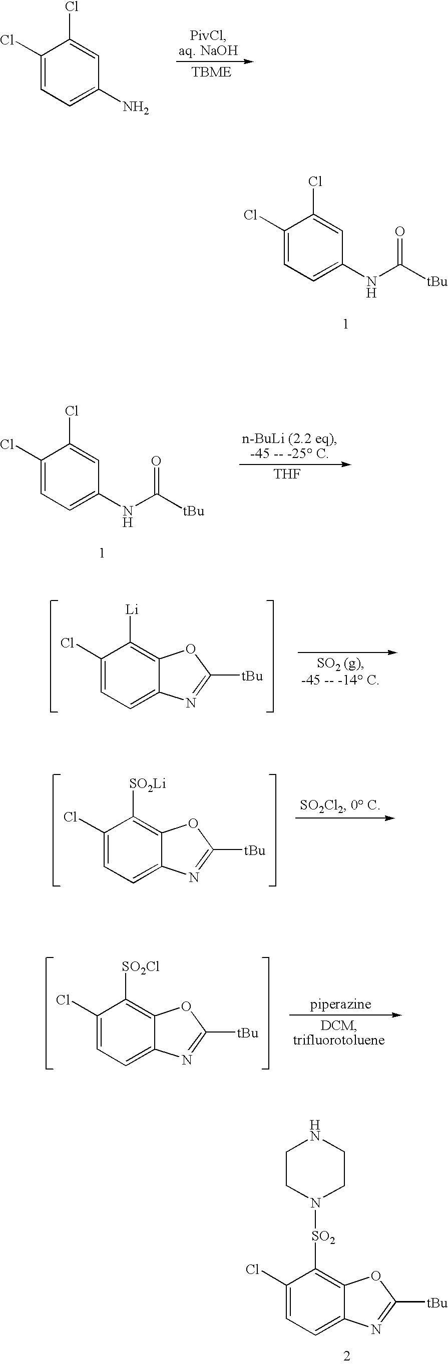

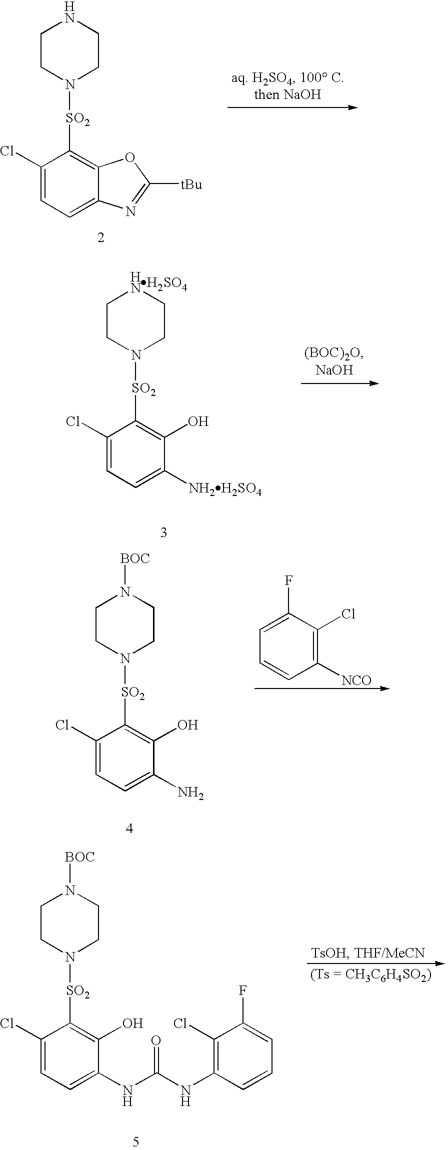

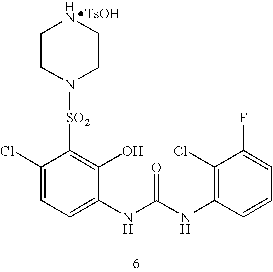

### 2a) Preparation of Compound 1

3,4-dichloroaniline (100 g) was dissolved in TBME (660 mL) and cooled to 10-15° C. Sodium hydroxide (94 g of a 30% aqueous solution) was added, and the solution stirred vigorously via mechanical stirrer. Trimethylacetyl chloride (84 mL) was added at such a rate as to keep the internal temperature below 35° C. When the addition was complete (10-15 min), the mixture was maintained at 30-35° C. for about 30 min, and then cooled to 0-5° C. over 30-40 minutes. The reaction mixture was held at 0-5° C. for 1 hr, and then filtered, rinsing first with 90:10 water/methanol (400 mL) and then water (600 mL.) Drying at 50-55° C. under vacuum afforded product as off-white crystals. A yield of 127 g was obtained.

### 2b) Preparation of Compound 2

A solution of Compound 1 (50 g) in tetrahydrofuran (300 mL) was cooled to −50-−40° C. under an inert atmosphere of nitrogen. N-Butyl lithium (2.5M in hexanes, 179 mL) was added at such a rate as to keep the solution's internal temperature between −45-−30° C. (ca. 15-30 min addition). The solution was held at ca. −35-−25° C. until HPLC indicated that the initial reaction was complete. The solution was then recooled to −45-−40° C., and sulfur dioxide (˜16.9 g) was bubbled through the solution, keeping the internal temperature below approximately −14° C., until the solution was acidic. When the reaction was complete, the mixture was warmed to −10-0° C. Starting at −2-3° C., sulfuryl chloride (25.2 mL) was then added dropwise to the tetrahydrofuran solution over 5-15 min, keeping the temperature below approximately 22° C. After 5 min, HPLC confirmed reaction completion, while the solution was kept around 10-15° C. The mixture was solvent-exchanged into α,α,α-trifluorotoluene under reduced pressure, filtered, partially concentrated under vacuum (to ˜100 mL), followed by addition of dichloromethane (350 mL). To this mixture was added a solution of piperazine (61.2 g) in dichloromethane (625 mL) at ambient temperature dropwise, keeping the solution's internal temperature at 15-27° C. (2 h addition). The reaction was held at 20-24° C. until complete. The mixture was washed with deionized water (200 mL), the organic layer concentrated, followed by addition of heptane (450 mL). The product (70.5 g) was isolated by filtration, washed with heptane (50-100 mL), and dried under vacuum at 50-55° C.

### 2c) Preparation of Compound 3

Compound 2 (30 g) was added to ˜16% (w/w in water) sulfuric acid (300 mL). The resulting mixture was heated to reflux at 99-103° C. for ˜6 hours. Upon completion of the reaction, the solution was cooled to 40-50° C., then concentrated to ˜60 mL under reduced pressure. Acetonitrile (225 mL) was added and the resulting suspension stirred at 20-25° C. for ˜1 hour. The product was isolated by filtration, washed with acetonitrile (135 mL) and dried at 45-50° C. under vacuum. A yield of 33.34 g was obtained.

### 2d) Preparation of Compound 4

Compound 3 (20 g) was added to deionized water (200 mL). The pH of the resulting solution was adjusted to 6.5-7.0 by adding 50% aq. sodium hydroxide (˜6.35 mL) while maintaining the internal temperature between 20-30° C. Then a solution of di-tert-butyl dicarbonate (8.9 g) in ethyl acetate (80 mL+20 mL rinse) was added. The pH of the resulting mixture was adjusted to 6.8-7.0 by adding 50% aq. sodium hydroxide (2.45 mL) while maintaining the internal temperature between 20-30° C. Upon completion of the reaction, the reaction solution is filtered to remove the small amount of precipitate. The two layers of the filtrate were separated, and the aqueous layer was extracted with ethyl acetate (140 mL). Combined ethyl acetate layers are washed with water (40 mL) and concentrated to 100 mL. Heptane (100 mL) was added and the resulting suspension was concentrated to 60 mL. This process was repeated once more. Heptane (140 mL) was then added, and the resulting suspension was stirred at 20-25° C. for ˜1 hour. The product was isolated by filtration, washed with heptane (80 mL) and dried at 40-45° C. under vacuum. A yield of 15.3 g was obtained.

### 2e) Preparation of Compound 5

Compound 4 (10 g) was added to dimethylformamide (20 mL) and acetonitrile (80 mL). 2-Chloro-3-fluorophenyl isocyanate (4.77 g) was added while maintaining the internal temperature between 20-30° C., followed by 10 mL acetonitrile rinse. The resulting mixture was stirred at 20-25° C. for ˜2 hours. Upon completion of the reaction, methanol (50 mL) was added. The resulting suspension was stirred at 20-25° C. for ˜10 minutes. Deionized water (150 mL) was added, and the resulting suspension stirred at 20-25° C. for ˜1 hour. The product was isolated by filtration, washed with deionized water (100 mL) and methanol (15-20 mL), and then dried at 40-45° C. under vacuum. A yield of 14.15 g was obtained.

### 2f) Preparation of Compound 6

### Procedure 1

Compound 5 (50 g) was dissolved in tetrahydrofuran (THF, 200 mL) and heated to 33-37° C. and held at 33-37° C. In another reactor, a solution of acetonitrile (250 mL), THF (50 mL) and p-toluenesulfonic acid monohydrate (43.9 g) was prepared. The resulting solution was heated to 33-37° C. and held at 33-37° C. The p-toluenesulfonic acid solution was filtered and transferred into the reactor containing Compound 5 and THF while maintaining the temperature at 33-37° C. After the starting material was consumed, micronized seeds of product (0.5 g) were charged in a minimal amount of acetonitrile (5 mL). The reaction mixture was then heated to 53-57° C. over ˜40 minutes, and held at that temperature for at least 4 hours. The reaction was cooled to 0-5° C., the product isolated by filtration, washed with acetonitrile (250 mL), and dried under vacuum at 55-60° C. A yield of 52.24 g was obtained.

### 2f) Preparation of Compound 6

### Procedure 2

Compound 5 (500 g) was charged to reactor 1 followed by acetonitrile (CAN, 3750 mL) and tetrahydrofuran (THF, 1250 mL). The solution was then heated to 60-65° C. and once a clear solution is observed, a clarifying filtration is performed to reactor 2. To reactor 1, p-toluenesulfonic acid monohydrate (TsOH·H₂O, 439 g) is added followed by ACN (750 mL) and THF (250 mL). The mixture was heated to 40-45° C. and once a clear solution was observed, a clarifying filtration was performed, adding the solution to reactor 2 (containing the starting material solution) and maintaining the temperature in reactor 2 at 50-60° C. The mixture was heated to reflux, and held at 70-80° C. until the reaction was complete. ˜3500 mL of solvent was removed by atmospheric distillation. The reactor was then charged with 2.5 L water followed by 4 L ACN, and the temperature adjusted to 70-80° C. After dissolution was observed, the resulting solution was cooled to 64-68° C. After 5-10 minutes, milled product Form III seeds (5 g) were added in a minimal amount of acetonitrile, and held at 64-68° C. for one hour. The mixture was cooled to 0-5° C. over 2 hours and held at 0-5° C. for ˜30 minutes before isolating the product by filtration. The solid product was washed with 2.5 L of acetonitrile, and dried under vacuum at 50±60° C. A yield of 480 g was obtained.

The present method makes use of compounds which inhibit chemokine function, in particular GROα, GROβ, GROγ, NAP-2 and ENA-78, and are useful in the treatment of endometriosis.

As used herein, the term “chemokine” refers to any secreted polypeptide that affects the functions of cells and is a molecule which modulates interactions between cells in the immune, inflammatory or hematopoietic response. A chemokine is primarily secreted through cell transmembranes and causes chemotaxis and activation of specific white blood cells and leukocytes, neutrophils, monocytes, macrophages, T-cells, B-cells, endothelial cells and smooth muscle cells. Examples of chemokines include, but are not limited to IL-8, GRO-α, GRO-β, GRO-γ, NAP-2, ENA-78, IP-10, MIP-1α, MIP-β, PF4, and MCP 1, 2, and 3.

In order to use a present compound in therapy, it will normally be formulated into a pharmaceutical composition in accordance with standard pharmaceutical practice.

The present compounds and pharmaceutical compositions incorporating such may conveniently be administered by any of the routes conventionally used for drug administration, for instance, orally, topically, or parenterally, preferably orally. The present compounds may be administered in conventional dosage forms prepared by combining with standard pharmaceutical carriers according to conventional procedures.

The present compounds may also be administered in conventional dosages in combination with a known, second therapeutically active compound. These procedures may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof.

The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time delay material well known to the art, such as glyceryl mono-stearate or glyceryl distearate alone or with a wax.

A wide variety of pharmaceutical forms can be employed. Thus, if a solid carrier is used, the preparation can be tableted, placed in a hard gelatin capsule in powder or pellet form or in the form of a troche or lozenge. The amount of solid carrier will vary widely but preferably will be from about 25 mg to about 1 g. When a liquid carrier is used, the preparation will be in the form of a syrup, emulsion, soft gelatin capsule, sterile injectable liquid such as an ampule or nonaqueous liquid suspension.

The present compounds may be administered topically, that is by non-systemic administration. This includes the application of a present compound externally to the epidermis, such that compound does not significantly enter the blood stream.

In contrast, systemic administration refers to oral, intravenous, intraperitoneal and intramuscular administration.

Formulations suitable for topical administration include liquid or semi-liquid preparations suitable for penetration through the skin to the site of inflammation such as liniments, lotions, creams, ointments or pastes, and drops. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

Lotions according to the present invention include those suitable for application to the skin. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel.

The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

The present compounds may be administered parenterally, that is by intravenous, intramuscular, subcutaneous, intranasal, intrarectal, intravaginal or intraperitoneal administration. Appropriate dosage forms for such administration may be prepared by conventional techniques.

For all methods of use disclosed herein for the present compounds the daily oral dosage regimen will preferably be from about 0.01 to about 80 mg/kg of total body weight. The daily parenteral dosage regimen about 0.001 to about 80 mg/kg of total body weight. The daily topical dosage regimen will preferably be from 0.1 mg to 150 mg, administered one to four, preferably two or three times daily. It will also be recognized by one of skill in the art that the optimal quantity and spacing of individual dosages of a present compound will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a present compound of or a pharmaceutically acceptable salt thereof given per day for a defined number of days, can be ascertained by those skilled in the art using conventional course of treatment determination tests.

The invention will now be described by reference to the following biological examples, which are merely illustrative and are not to be construed as a limitation of the scope of the present invention.

### Biological Examples

The compounds used in the methods of the present invention are tested in the following assays.

### Receptor Binding Assay:

[¹²⁵I] IL-8 (human recombinant) was obtained from Amersham Corp., Arlington Heights, Ill., with specific activity 2000 Ci/mmol. GRO-α was obtained from NEN—New England Nuclear. All other chemicals were of analytical grade. High levels of recombinant human IL-8 type α and β receptors were individually expressed in Chinese hamster ovary cells as described in Holmes, et al., Science, 1991, 253, 1278, incorporated herein to the extent required to perform the present assay. The Chinese hamster ovary membranes were homogenized according to Haour, et al., J. Biol. Chem., 249 pp 2195-2205 (1974), incorporated herein to the extent required to perform the present assay, except that the homogenization buffer was changed to 10 mM Tris-HCL, 1 mM MgSO₄, 0.5 mM EDTA (ethylene-diaminetetra-acetic acid), 1 mM PMSF (α-toluenesulphonyl fluoride), 0.5 mg/L Leupeptin, pH 7.5.

Membrane protein concentration was determined using Pierce Co. micro-assay kit using bovine serum albumin as a standard. All assays were performed in a 96-well micro plate format. Each reaction mixture contained ¹²⁵I IL-8 (0.25 nM) or ¹²⁵I GRO-α and 0.5 μg/mL of IL-8Rα or 1.0 μg/mL of IL-8Rβ membranes in 20 mM Bis-Trispropane and 0.4 mM Tris HCl buffers, pH 8.0, containing 1.2 mM MgSO₄, 0.1 mM EDTA, 25 mM Na and 0.03% CHAPS. In addition, the compound of interest was added which had been pre-dissolved in DMSO so as to reach a final concentration of between 0.01 nM and 100 uM. The assay was initiated by addition of ¹²⁵I-IL-8. After 1 hour at room temperature, the plate was harvested using a Tomtec 96-well harvester onto a glass fiber filtermat blocked with 1% polyethylenimine/0.5% BSA and washed 3 times with 25 mM NaCl, 10 mM TrisHCl, 1 mM MgSO₄, 0.5 mM EDTA, 0.03% CHAPS, pH 7.4. The filter was then dried and counted on the Betaplate liquid scintillation counter. The recombinant IL-8 Rα, or Type I, receptor is also referred to herein as the non-permissive receptor and the recombinant IL-8 Rβ, or Type II, receptor is referred to as the permissive receptor.

An IC₅₀ value of <10 uM was considered active in the present assay. The Example 1 compound had an IC₅₀ of about 13 nM.

### Rat Model of Estrogen Opposition: Uterotrophic Model Assay

A rat estrogen opposition model (Lundeen, S. G. et al., (2001) Rat Uterine Complement C3 Expression as a Model for Progesterone Receptor Modulators: Characterization of the New Progestin Trimegestone. J. Steroid Biochem. & Mol. biol. 78, 137-143, incorporated herein to the extent required to perform the present assay) was used to determine the efficacy of Example 2f at 30 mg/kg/day. This was a 48 hour rodent model involving a pre-treatment of compound followed by two days of combined estrogen (0.08 mg/kg) and compound treatment. At termination, uteri were removed from the rats weighed and sectioned for downstream biochemical analysis. Estrogen in this model was pro-inflammatory, inducing edema and infiltration of neutrophils into the endometrium causing a 4 to 5 fold increase in uterine weight. A significant effect of compound treatment on uterine weight was considered approximately a 25% decrease. The effect of compound treatment was also monitored by the detection of complement 3 (C3) gene expression and the FBJ murine osteosarcoma viral oncogene homologs (cfos), which were both upregulated by estrogen stimulation in the uterus. The gene expression of matrix metallo protease 9 (MMP9) was also measured, as this has been shown to be significantly modulated by CXCR2 activation (Arici, A., Uterine Chemokines in Reproductive Physiology and Pathology, American Journal of reproductive Immunology 47 (4), 213-221, incorporated herein to the extent required to perform the present assay).

Example 2f at 30 mg/kg/day significantly reduced estrogen-induced uterine wet weight (66%+/−5%). The gene expression of C3 and cfos were significantly reduced compared to estrogen only treatment by 84% and 50%, respectively. The gene expression level of MMP-9 was significantly reduced by 50% compared to vehicle control and estrogen treated rats. These results indicate that Example 2f inhibited proliferation and protease-induced remodeling in estrogen-driven uterine proliferative diseases, such as endometriosis.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

1. A method of treating endometriosis in a woman in need thereof which comprises administering to said woman an effective amount of a compound which is N-[4-chloro-2-hydroxy-3-(piperazine-1-sulfonyl)phenyl]-N′-(2-chloro-3-fluorophenyl)urea or a pharmaceutically acceptable salt thereof.

2. The method according to claim 1 wherein the compound is N-[4-chloro-2-hydroxy-3-(piperazine-1-sulfonyl)phenyl]-N′-(2-chloro-3-fluorophenyl)urea hydrochloride.

3. The method according to claim 1 wherein the compound is N-[4-chloro-2-hydroxy-3-(piperazine-1-sulfonyl)phenyl]-N′-(2-chloro-3-fluorophenyl)urea p-toluenesulfonate.

4. The method according to claim 1 wherein the compound is administered orally.

5. A method of preventing endometriosis in a woman at higher than normal risk of developing endometriosis which comprises administering to said woman an effective amount of a compound which is N-[4-chloro-2-hydroxy-3-(piperazine-1-sulfonyl)phenyl]-N′-(2-chloro-3-fluorophenyl)urea or a pharmaceutically acceptable salt thereof.

6. The method according to claim 5 wherein the compound is N-[4-chloro-2-hydroxy-3-(piperazine-1-sulfonyl)phenyl]-N′-(2-chloro-3-fluorophenyl)urea hydrochloride.

7. The method according to claim 5 wherein the compound is N-[4-chloro-2-hydroxy-3-(piperazine-1-sulfonyl)phenyl]-N′-(2-chloro-3-fluorophenyl)urea p-toluenesulfonate.

8. The method according to claim 5 wherein the compound is administered orally.

9.-14. (canceled)
